(19) United States
(12) Patent Application Publication
(10) Pub. No.: US 2022/0048939 A1
(43) Pub. Date: Feb. 17, 2022

(54) AROMATIC RING COMPOUND

(71) Applicants: Lin XU, Kunming, Yunnan (CN); HAIKOU KAIBAO INDUSTRIAL CO., LTD, Haikou, Hainan (CN); SURELY BIOPHARMACEUTICAL TECHNOLOGY CO., LTD, Kunming, Yunnan (CN)

(72) Inventors: Lin XU, Kunming, Yunnan (CN); Jianfeng LI, Kunming, Yunnan (CN); ChunShan TANG, Kunming, Yunnan (CN); Jingfei HUANG, Kunming, Yunnan (CN); Chunhong JIANG, Kunming, Yunnan (CN); Qixin ZHOU, Kunming, Yunnan (CN)

(21) Appl. No.: 17/428,184

(22) PCT Filed: Apr. 21, 2020
(86) PCT No.: PCT/CN2020/085987

§ 371 Date: Aug. 3, 2021

(30) Foreign Application Priority Data
Aug. 21, 2019 (CN) 201910775698.7

(51) Int. Cl. C07H 17/02; C07H 15/203; C07H 1/00; A61K 45/06; A61K 9/00; A61P 25/24

(52) U.S. Cl. CPC C07H 17/02; C07H 15/203; C07H 1/00; C07B 2200/05; A61K 9/0053; A61P 25/24; A61K 45/06

(57) ABSTRACT

A novel aromatic ring compound is represented by general formula (I). This compound, isomer, prodrug, solvate, pharmaceutically acceptable salt and pharmaceutical composition thereof are useful in preparing medicaments for treating depression and related symptoms.

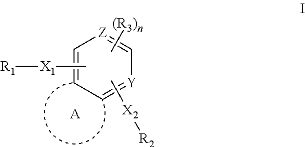

### CROSS REFERENCES TO RELATED APPLICATIONS

The present application is a U.S. national stage entry of International Application No. PCT/CN2020/085987, filed Ap. 21, 2020, which claims the priority Chinese Patent Application No. 201910775698.7 filed to the China National Intellectual Property Administration on Aug. 21, 2019 and entitled “AROMATIC RING COMPOUND”, which is incorporated herein by reference in its entirety.

### TECHNICAL FIELD

The present invention relates to a novel aromatic ring compound, an isomer, a prodrug, a solvate, a pharmaceutically acceptable salt and an isotopically labeled compound thereof, and a pharmaceutical composition thereof, and use of preparing a medicament for treating depression and related symptoms.

### BACKGROUND

Depression is a common mental disease, and is listed in top ten diseases due to its high incidence, high disability rate and high suicide rate. The burden of depression in developed countries ranks the top in the list of burdens of illness and disability.

Currently, the major treatments of depression are antidepressants of monoamine pathway. Since the mechanisms of action for antidepressants are similar, the antidepressants have similar efficacy and toxic and side effects, for example: efficacy in about 2/3 of the depression patients, delayed action until 4-8 weeks of continuous administration, increase suicide risk at early stage of treatment, andside effects such as gastrointestinal dysfunction.

There is an urgent need in the medical field for discovering and developing new antidepressants with new molecular structures and new mechanisms of action.

### SUMMARY

The present invention provides a novel aromatic ring compound as a novel antidepressant with a novel molecular structure and a novel mechanism of action, an isomer, a prodrug, a solvate, a pharmaceutically acceptable salt and an isotopically labeled compound thereof, and a pharmaceutical composition thereof, and use of the same in preparing a medicament fortreating depression and related symptoms, which features fast action, good safety and mild side effect.

In the first aspect of the present invention, an aromatic ring compound of formula I, an isomer, a prodrug, a solvate, a pharmaceutically acceptable salt or an isotopically labeled compound thereof is provided,

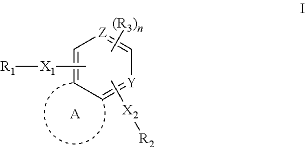

wherein,

R₁ and R₂ each independently represent H or a saccharide unit, and at least one of R₁ and R₂ is a saccharide unit; the saccharide unit may be selected from C₄₋₆ monosaccharide such as glucose, mannose, allose, galactose, arabinose and xylose, or may be selected from disaccharides and higher-order oligosaccharide such as sucrose, lactose, cellobiose and maltose, wherein carbon and oxygen atoms on the saccharide unit may be optionally substituted with sulfur, nitrogen or carbon;

when R₁ and R₂ each independently represent H, the —X₁— and —X₂— to which they are connected respectively represent —O—, —S— or a bond;

when R₁ and R₂ each independently represent a saccharide unit, the —X₁— and —X₂— to which they are connected respectively represent glycosidic bond formed by the saccharide unit and a non-saccharide unit (aromatic aglycon) and each independently represent O, S , N or a bond (i.e., O-glycosidic bond, S-glycosidic bond, N-glycosidic bond, or C-glycosidic bond is formed); or —X₁ — and —X₂— are —CH₂—;

Y and Z each independently represent C, O, N, S, P or Si;

R₃ represents hydrogen, hydroxyl, or a substituted or unsubstituted C₁-C₂₀ aliphatic hydrocarbyl; n is selected from 1, 2, 3, 4 and 5; the aromatic ring may be

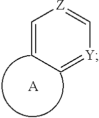

(with absence of ring A) or

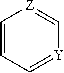

ring A may be a C₆₋₁₀ aryl, a C₃₋₈ cycloalkyl, a 3-10 membered heterocycloalkyl, or a 5-12 membered heteroaryl.

According to one embodiment of the present invention, ring A may be phenyl ring, a 5-6 membered heteroaryl, a C₅₋₆ cycloalkyl or a 5-6 membered heterocycloalkyl; in ring A, a heteroatom, if present, may be O, S or N; ring A may be, for example, phenyl ring, cyclopentane, cyclohexane, or a nitrogen- or oxygen-containing 5-6-membered heterocyclic ring;

the C₁-C₂₀ aliphatic hydrocarbyl may be a saturated hydrocarbyl or an unsaturated hydrocarbyl, for example, selected from a C₁-C₂₀ alkyl, a C₂-C₂₀ alkenyl and a C₂-C₂₀ alkynyl, and specifically, selected from a (C₁-C₆) alkyl, a (C₂-C₆) alkenyl and a (C₂-C₆) alkynyl;

the C₁-C₂₀ aliphatic hydrocarbyl can be further substituted, and the “substituted C₁-C₂₀ aliphatic hydrocarbyl” may be a C₁-C₂₀ aliphatic hydrocarbyl containing one, two or more halogen and/or oxygen, sulfur, nitrogen, phosphorus atoms; for example, a halogenated (C₁-C₆) alkyl, a halogenated (C₁-C₆) alkoxy, or a (C₁-C₆) alkoxy, and specifically, CF₃, CHF₂, and OCH₃; for example, a C₁-C₂₀ aliphatic hydrocarbyl substituted with hydroxyl, amino, carboxyl, fluorine, trifluoromethyl, difluoromethyl, formyl, or phosphate, sulfate, phosphate or sulfonate group; the halogen is selected from F, Cl, Br and I;

the saccharide unit is preferably glucose, mannose, allose, galactose, arabinose or xylose; the saccharide unit may be in the D configuration or L configuration;

According to one embodiment of the present invention, the configurations of the glycosidic bonds formed by the saccharide unit and the aromatic aglycon are independently selected from an a configuration and a β configuration, preferably a β configuration; the glycosidic bond may be formed by connecting the aglycon to the Cl position of the ring moiety of the saccharide unit;

the aromatic ring may be phenyl ring,

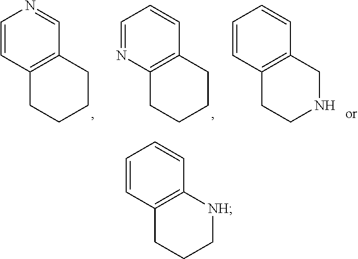

in the isotopically labeled compound, the isotopically labeled atoms include, but are not limited to, hydrogen, carbon, nitrogen, oxygen and phosphorus, as they can be substituted by isotopically labeled atoms ²H, ³H, ¹¹C, ¹³C, ¹⁴C, ¹⁵N, ³¹P, ³²P and ³⁵S.

According to one embodiment of the present invention, the pharmaceutically acceptable salt may be, for example, an acid addition salt of the compound of the present invention having a nitrogen atom in the chain or ring with sufficient basicity, for example, an acid addition salt formed with the following inorganic acids: hydrochloric acid, hydrofluoric acid, hydrobromic acid, hydroiodic acid, sulfuric acid, pyrosulfuric acid, phosphoric acid or nitric acid; a bisulfate; or an acid addition salt formed with the following organic acids: formic acid, acetic acid, acetoacetic acid, pyruvic acid, trifluoroacetic acid, propionic acid, butyric acid, caproic acid, enanthic acid, undecanoic acid, lauric acid, benzoic acid, salicylic acid, 2-(4-hydroxybenzoyl)benzoic acid, camphoric acid, cinnamic acid, cyclopentanepropionic acid, digluconic acid, 3-hydroxy-2-naphthoic acid, nicotinic acid, pamoic acid, pectinic acid, persulfuric acid, 3-phenylpropionic acid, picric acid, pivalic acid, 2-hydroxyethanesulfonic acid, itaconic acid, sulfamic acid, trifluoromethanesulfonic acid, dodecylsulfuric acid, ethanesulfonic acid, benzenesulfonic acid, p-toluenesulfonic acid, methanesulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, citric acid, D-gluconic acid, mandelic acid, ascorbic acid, glucoheptonic acid, glycerophosphoric acid, aspartic acid, sulfosalicylic acid, hemisulfuric acid or thiocyanic acid.

In addition, another suitable pharmaceutically acceptable salt of the compound of the present invention with sufficient acidity is an alkali metal salt (e.g., a sodium or potassium salt), an alkaline earth metal salt (e.g., a calcium or magnesium salt), an ammonium salt, or a salt formed with an organic base which affords a physiologically acceptable cation, for example, a salt formed with the following substance: sodium ion, potassium ion, N-methylglucamine, dimethylglucamine, ethylglucamine, lysine, dicyclohexylamine, 1,6-hexanediamine, ethanolamine, glucosamine, meglumine, sarcosine, serinol, tris(hydroxymethyl)aminomethane, aminopropanediol or 1-amino-2,3,4-butanetriol. As an example, the pharmaceutically acceptable salt includes salts formed with —COOH group and the following substance: sodium ion, potassium ion, calcium ion, magnesium ion, N-methylglucamine, dimethylglucamine, ethylglucamine, lysine, dicyclohexylamine, 1,6-hexanediamine, ethanolamine, glucosamine, meglumine, sarcosine, serinol, tris(hydroxymethyl)aminomethane, aminopropanediol or 1-amino-2,3,4-butanetriol.

Preferably, when R₃ is a C₁-C₂₀ aliphatic hydrocarbyl containing an amino functional group or the aromatic ring is a nitrogen-containing heterocyclic ring, the compound can form a pharmaceutically acceptable salt with an acid. Preferably, the acid is selected from sulfuric acid, phosphoric acid, methanesulfonic acid, p-toluenesulfonic acid, benzenesulfonic acid, citric acid, oxalic acid, lactic acid, acetic acid, succinic acid, any one of 20 natural L-amino acids and corresponding D-amino acids thereof, and an oxygen-free acid; the oxygen-free acid may be HCl, HBr, HI or HF.

Preferably, for the aromatic ring compound, the isomer, the prodrug, the solvate, the pharmaceutically acceptable salt or the isotopically labeled compound thereof, the aromatic ring compound is selected from the following formulas Ia, Ib, Ic, Id, Ie, If, Ig, Ih, Ii and Ij:

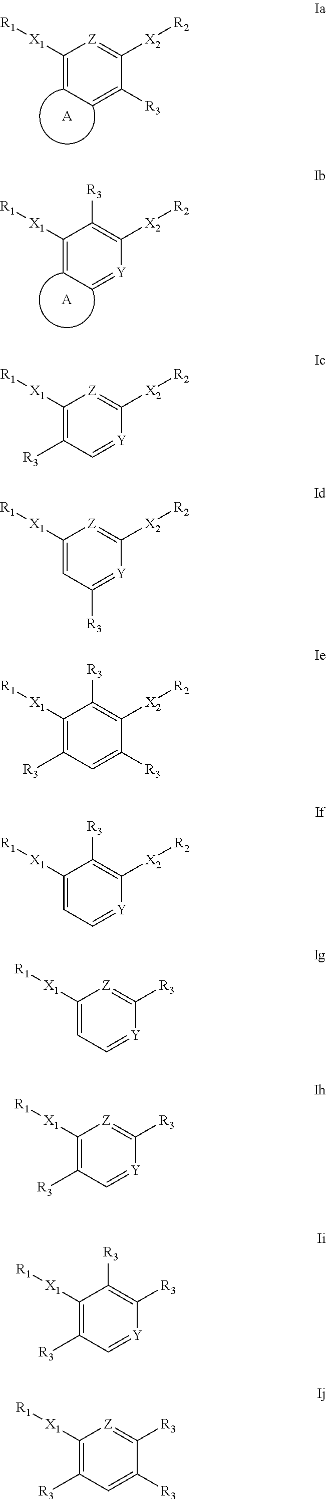

In formulas Ia, Ib, Ic, Id, Ie, If, Ig, Ih, Ii and Ij, R₁, R₂, R₃, ring A, X₁, X₂, Y and Z are as defined in formula I; preferably, for the aromatic ring compound, the isomer, the prodrug, the solvate, the pharmaceutically acceptable salt or the isotopically labeled compound thereof, the aromatic ring compound is selected from the following formulas IIa, IIb, IIc, IId, IIe, IIf, IIg, IIh and Ili:

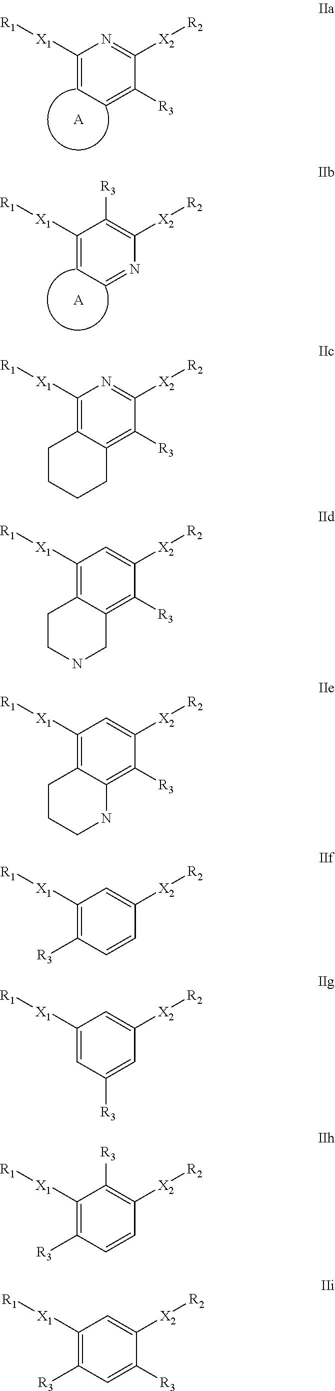

In formulas IIa, IIb, IIc, IId, IIe, IIf, IIg, IIh and IIi, R₁, R₂, R₃, ring A, X₁ and X₂ are as defined in formula I;

Preferably, for the aromatic ring compound, the isomer, the prodrug, the solvate, the pharmaceutically acceptable salt or the isotopically labeled compound thereof, the aromatic ring compound is selected from:

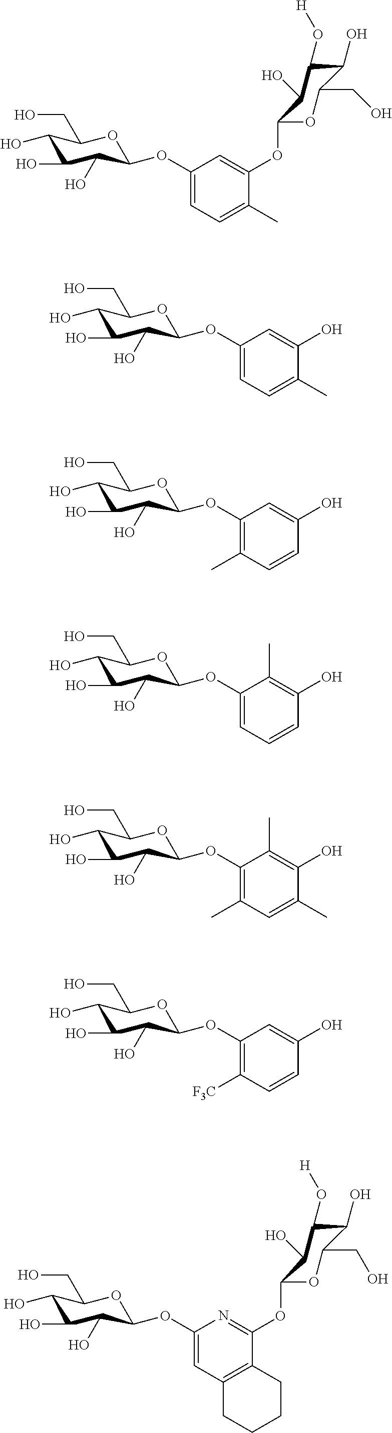

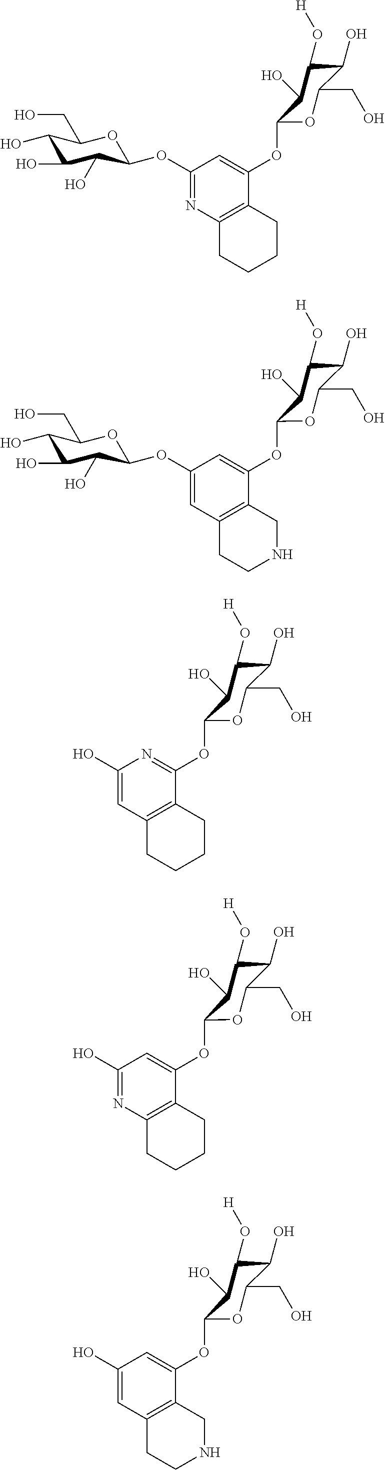

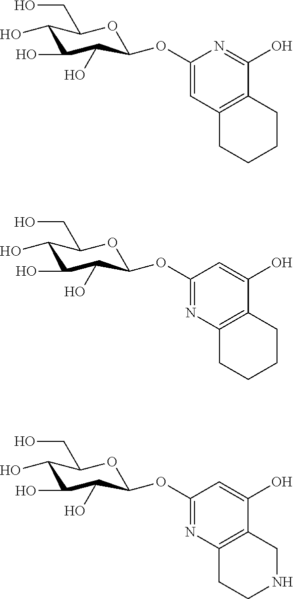

In the second aspect of the present invention, a pharmaceutical composition is provided, comprising the aromatic ring compound of formula I, the isomer, the prodrug, the solvate, the pharmaceutically acceptable salt or the isotopically labeled compound thereof, and a pharmaceutically acceptable carrier.

In the third aspect of the present invention, use of the aromatic ring compound of formula I, the isomer, the prodrug, the solvate, the pharmaceutically acceptable salt or the isotopically labeled compound thereof, or the pharmaceutical composition thereof in preparing a medicament for treating related diseases by positively regulating NMDA receptors, preferably use of the same in treating depressive disorders, is provided.

The depressive disorders include depression and other mental diseases or symptoms closely related to clinical symptoms of depression, such as bipolar depression, affective cognitive dysfunction, anxiety, autism, obsessive-compulsive disorder, sleep disorder, anorexia, suicidal or self-mutilation ideation or behavior, schizophrenia, senile mental disorder, and depression symptoms in senile dementia patients. Preferably, the aromatic ring compound, the isomer, the prodrug, the solvate, the pharmaceutically acceptable salt or the isotopically labeled compound thereof, or the pharmaceutical composition is used alone or in combination with other therapeutic agents for treating nerve damage and depressive disorders.

In the fourth aspect of the present invention, a pharmaceutical formulation is provided, comprising the aromatic ring compound, the isomer, the prodrug, the solvate, the pharmaceutically acceptable salt or the isotopically labeled compound thereof, and further comprising a pharmaceutically acceptable carrier.

Herein, the carrier in the pharmaceutical composition/formulation is “acceptable” in that it is compatible with (and preferably, capable of stabilizing) the active ingredient of the composition and is not deleterious to the subject being treated. One or more solubilizers may be used as pharmaceutical excipients for delivery of the active compound.

In some embodiments, the compound of the present invention or pharmaceutical composition/formulation containing the same may be administered orally, i.e., may be in any acceptable dosage forms for oral administration, including capsule, tablet, emulsion, aqueous suspension, suppository, spray, inhalation, dispersion and solution.

In some embodiments, the compound of the present invention or pharmaceutical composition/formulation containing the same is administered by methods including, but not limited to, oral administration. Such methods are known to those skilled in the art, for example transdermal, inhalational, transmucosal nasal, topical, intrathecal, ophthalmic, internal, intracerebral, rectal, sublingual, buccal, intraurethral, and parenteral administrations (the term “parenteral” including subcutaneous, intradermal, intravenous, intramuscular, intraarticular, intraarterial, intrasynovial, intrasternal, intrathecal, intralesional and intracranial injections or infusions). For example, vascular injections may, for example, be administered intravenously, intraarterially or subcutaneously. The administration may be continuous or intermittent. Preferably, the formulation is selected from an injection, an oral capsule and tablet, and other conventional dosage forms.

The subject to which the formulation is administered includes human or animals; the animals include rodents, rabbit, dog, pig, cat, or non-human primates; the rodents include murines.

Preferably, when the subject is an animal, the formulation is administered in a daily dose of 1.0-30 mg/kg, preferably 5.0-30.0 mg/kg, for example 5.0 mg/kg, 10.0 mg/kg, 15.0 mg/kg, 20.0 mg/kg, or 25.0 mg/kg, on kilogram body weight basis.

Preferably, the dose for human is 45-90 mg/subject (60 kg body weight)/day, preferably 50-60 mg/subject (60 kg body weight)/day, on body surface area basis.

In the fifth aspect of the present invention, a method for using the aromatic compound of formula I, the isomer, the prodrug, the solvate, the pharmaceutically acceptable salt or the isotopically labeled compound thereof, or the pharmaceutical composition alone or in combination with other therapeutic agents for treating nerve damage and depressive disorders is provided.

Preferable conditions for the aromatic ring compound, the isomer, the prodrug, the solvate, the pharmaceutically acceptable salt or the isotopically labeled compound thereof according to the first aspect are also applicable to the second to fifth aspects.

In the sixth aspect of the present invention, the following compounds are provided:

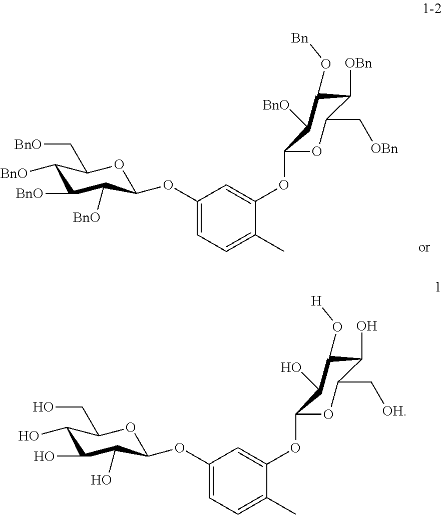

In the seventh aspect of the present invention, a method for preparing the aromatic ring compound of formula I is provided, comprising: condensing a hydroxyl-protected saccharide starting material with an aglycon, followed by deprotecting to give a product; the method further may comprise a post-treatment procedure.

In the eighth aspect of the present invention, a method for preparing the compound of formula 1-2 is provided. The reaction scheme is as follows:

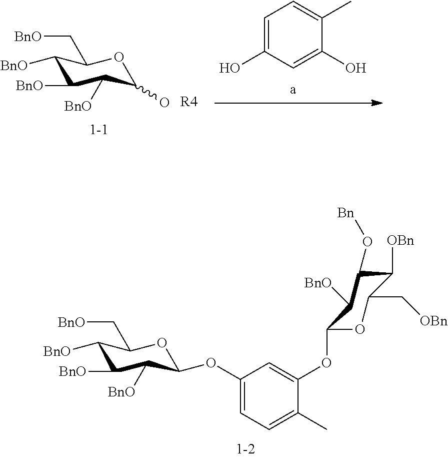

wherein R₄ is selected from hydrogen and an isotopically-labeled atom thereof (e.g., ²H, ³H).

The preparation method comprises the following procedure: subjecting a compound of formula 1-1 and compound a to Mitsunobu reaction to give a compound of formula 1-2.

A trihydrocarbylphosphine, compound a and a compound of formula 1-1 are dissolved in a first organic solvent. The reaction system is cooled to −15 to 0° C., added with an azodicarboxylate ester and stirred before the temperature is raised to 20 to 50° C. The system is then stirred until the starting materials disappear, and concentrated, separated and purified.

Preferably, the trihydrocarbylphosphine is selected from: triphenylphosphine, tributylphosphine, tri(o-tolyl)phosphine, trimethylphosphine, triethylphosphine and tripropylphosphine.

Preferably, the azodicarboxylate ester is a C₁₋₁₀ azodicarboxylate ester, for example: DIAD (diisopropyl azodicarboxylate), DMAD (dimethyl azodicarboxylate), or DEAD (diethyl azodicarboxylate).

Preferably, the first organic solvent is selected from an ether solvent (such as diethyl ether, tetrahydrofuran), a halogenated hydrocarbon solvent (e.g., dichloromethane, trichloromethane) and an aromatic hydrocarbon solvent (e.g., toluene, benzene); preferably, the first organic solvent is tetrahydrofuran, dichloromethane, toluene or benzene;

Preferably, the separation and purification is silica gel column chromatography.

Preferably, the concentration is a concentration in vacuum.

Preferably, the molar ratio of compound a, the compound of formula 1-1, and the trihydrocarbylphosphine to the azodicarboxylate ester is 1:(1-5):(1-5):(1-5), more preferably, the molar ratio may be 1:(2-4):(2-4):(2-4), and still more preferably, 1:2.1:2.5:2.5.

Preferably, the temperature is raised to room temperature.

In the another aspect of the present invention, a method for preparing the compound of formula 1 is provided. The reaction scheme is as follows:

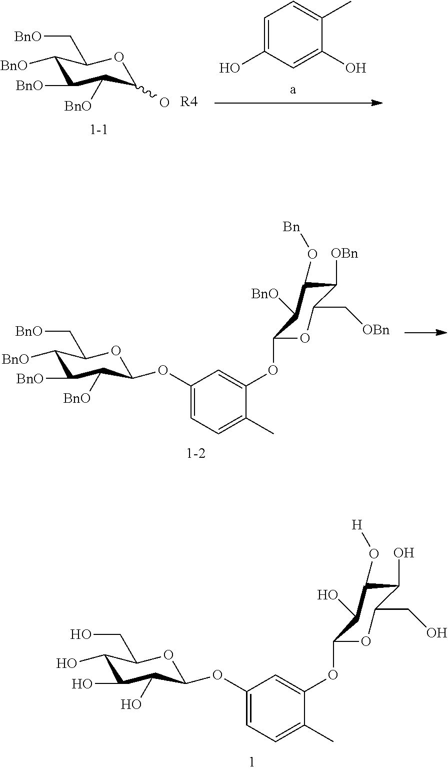

wherein, R₄ is selected from hydrogen and an isotopically-labeled atom thereof (e.g., ²H, ³H). The preparation method comprises the following procedures:

1) subjecting a compound of formula 1-1 and compound a to Mitsunobu reaction to give a compound of formula 1-2;

2) subjecting the compound of formula 1-2 to catalytic hydrogenolysis reaction to give a compound of formula 1.

Preferably, step 1) comprises the following:

a trihydrocarbylphosphine, compound a and a compound of formula 1-1 are dissolved in a first organic solvent. The reaction system is cooled to -15 to 0° C., added with an azodicarboxylate ester and stirred before the temperature is raised to 20 to 50° C. The system is then stirred until the starting materials disappear, and concentrated, separated and purified.

Preferably, the trihydrocarbylphosphine is selected from: triphenylphosphine, tributylphosphine, tri(o-tolyl)phosphine, trimethylphosphine, triethylphosphine and tripropylphosphine.

Preferably, the azodicarboxylate ester is a C₁₋₁₀ azodicarboxylate ester, for example: DIAD (diisopropyl azodicarboxylate), DMAD (dimethyl azodicarboxylate), or DEAD (diethyl azodicarboxylate).

Preferably, the first organic solvent is selected from an ether solvent (e.g., diethyl ether, tetrahydrofuran), a halogenated hydrocarbon solvent (e.g., dichloromethane, trichloromethane) and an aromatic hydrocarbon solvent (e.g., toluene, benzene); preferably, the first organic solvent is tetrahydrofuran, dichloromethane, toluene or benzene;

Preferably, the separation and purification is silica gel column chromatography.

Preferably, the concentration is a concentration in vacuum.

Preferably, the molar ratio of compound a, a compound of formula 1-1, and the trihydrocarbylphosphine to the azodicarboxylate ester is 1:(1-5):(1-5):(1-5), more preferably, the molar ratio may be 1:(2-4):(2-4):(2-4), and still more preferably, 1:2.1:2.5:2.5.

Preferably, the temperature is raised to room temperature.

Preferably, step 2) comprises the following: the compound of formula 1-2 is dissolved in a second organic solvent. The reaction system is added with a catalyst in inert gas atmosphere, stirred at 20 to 50° C. in hydrogen atmosphere at one standard atmosphere pressure until complete conversion of the starting materials. A post-treatment is performed.

Preferably, the second organic solvent is selected from a C₁-C₆ aliphatic alcohol and a C₁-C₆ alicyclic alcohol solvent, preferably methanol, ethanol, isopropanol or n-butanol.

Preferably, the post-treatment comprises that the reaction system is filtered, the filtrate is washed, and the solvent is removed from the filtrate. Preferably, the filtration is performed under reduced pressure, and/or , the filter cake is washed with the second organic solvent, and/or, the solvent is removed under reduced pressure.

Preferably, the catalyst is selected from palladium on carbon and palladium hydroxide on activated carbon. More preferably, the palladium hydroxide on activated carbon catalyst comprises 20 wt % of palladium hydroxide, and the palladium on carbon catalyst comprises 10 wt % of Pd.

The inert gas may be selected from nitrogen, helium and argon, preferably nitrogen.

Unless otherwise indicated, the definitions of groups and terms described in the specification and claims of the present application, including exemplary definitions, illustrative definitions, preferred definitions, definitions described in tables, definitions of specific compounds in the examples and the like, may be arbitrarily used in combination and conjugation with each other. Such definitions of groups and structures of compounds in combinations and conjugations should fall within the scope of the present application.

When a numerical range defined by “integer” is recited in the specification and claims of this application, it shall be construed as reciting both endpoints of the range and every integer within the range. For example, “an integer of 0 to 6” shall be construed to include every integer of 0, 1, 2, 3, 4, 5 and 6. The term “more” refers to three or more.

The term “saccharide unit”, may also be referred to as “glycon”. The saccharide unit may be defined as a residue of the complete saccharide molecular structure excluding any one or more of hydroxyl groups present in the structure having a possibility of forming a glycosidic bond. Furthermore, the saccharide unit may be conventionally linked to a non-saccharide unit moiety (aglycon) via a glycosidic bond, i.e., the saccharide unit corresponds to a residue of the complete saccharide molecular structure excluding the moiety (terminal hydroxyl group of the saccharide) forming a glycosidic bond (e.g., O-glycosidic bond, S-glycosidic bond, N-glycosidic bond, or C-glycosidic bond); likewise, the saccharide unit described herein may also be linked to a non-saccharide structure using other connecting groups commonly used for chemical modifications, e.g. —CH₂—.

The glycon/saccharide unit may be selected from monosaccharides such as glucose, mannose, allose, galactose, arabinose and xylose, or may be selected from disaccharides or higher-order oligosaccharides such as sucrose, lactose, cellobiose and maltose (i.e., the glycon/saccharide unit corresponds to the saccharide residue moiety of the monosaccharides, disaccharides or oligosaccharides). The saccharide unit may be further connected to a non-saccharide unit moiety by a connecting group such as CH₂.

The term “halogen” refers to F, Cl, Br and I. In other words, F, Cl, Br and I may be described as “halogen” in this specification.

The term “aliphatic hydrocarbyl” includes saturated or unsaturated, linear or branched chain hydrocarbon groups. The aliphatic hydrocarbyl may be selected from alkyl, alkenyl, alkynyl and the like. The number of carbon atoms of the aliphatic hydrocarbyl is selected from 1 to 20, preferably from 1 to 12, and more preferably from 1 to 6. Specifically, the aliphatic hydrocarbyl includes, but is not limited to: methyl, ethyl, n-propyl, isopropyl, n-butyl, isobutyl, tert-butyl, n-pentyl, isopentyl, neopentyl, n-hexyl, ethenyl, 1-propenyl, 2-propenyl, 1-methylethenyl, 1-butenyl, 1-ethylethenyl, 1-methyl-2-propenyl, 2-butenyl, 3-butenyl, 2-methyl-1-propenyl, 2-methyl-2-propenyl, 1-pentenyl, 1-hexenyl, ethynyl, 1-propynyl, 2-propynyl, 1-butynyl, 1-methyl-2-propynyl, 3-butynyl, 1-pentynyl and 1-hexynyl. The aliphatic hydrocarbyl may optionally comprise one or more other suitable substituents. Examples of such substituents may include hydroxyl, halogen, cyano, amino and other groups. For example, the aliphatic hydrocarbyl may contain one, two or more halogens, indicating that one, two or more hydrogen atoms of the aliphatic hydrocarbyl may be substituted with an equivalent number of halogens. If the aliphatic hydrocarbyl contains more than one carbon atoms, those carbons are not necessarily linked to each other. For example, at least two of the carbons may be linked via a suitable atom or group. That is, the aliphatic hydrocarbyl may optionally contain one, two or more heteroatoms (or may be construed as optional insertion of heteroatoms into the aliphatic hydrocarbyl group at any C—C bond or C—H bond). Suitable heteroatoms will be apparent to those skilled in the art and include, for example, sulfur, nitrogen, oxygen, phosphorus and silicon. The aliphatic hydrocarbyl containing heteroatom may be selected from, for example, the following groups: (C₁-C₆) aliphatic hydrocarbyl oxy, (C₁-C₆) aliphatic hydrocarbyl thiol, halogenated (C₁-C₆) aliphatic hydrocarbyl, halogenated (C₁-C₆) aliphatic hydrocarbyl oxy, halogenated (C₁-C₆) aliphatic hydrocarbyl thiol, (C₁-C₆) aliphatic hydrocarbyl oxy (C₁-C₆) aliphatic hydrocarbyl, (C₁-C₆) aliphatic hydrocarbyl thiol (C₁-C₆) aliphatic hydrocarbyl, N-(C₁-C₃) aliphatic hydrocarbyl amino (C₁-C₆) aliphatic hydrocarbyl, and N,N-di-(C₁-C₃) aliphatic hydrocarbyl amino (C₁-C₆) aliphatic hydrocarbyl; the aliphatic hydrocarbyl may also be selected from, for example, methoxymethyl, ethoxymethyl, propoxymethyl, methoxyethyl, ethoxyethyl, propoxyethyl, methoxypropyl, ethoxypropyl, propoxypropyl, N-methylaminomethyl, N-methylaminoethyl, N-ethylaminoethyl, N,N-dimethylaminomethyl, N,N-dimethylaminoethyl, and N,N-diethylaminoethyl; for example, the aliphatic hydrocarbyl may be CF₃, CHF₂ and OCH₃; for example, the aliphatic hydrocarbyl may be a C₁-C₂₀ aliphatic hydrocarbyl substituted with hydroxyl, amino, carboxyl, fluorine, trifluoromethyl, difluoromethyl, formyl, or phosphate, sulfate or sulfonate group. The “aliphatic hydrocarbyl” moiety contained in the other groups is as defined above.

The term “cycloalkyl” refers to a saturated or partially unsaturated (containing 1 or 2 double bonds) monocyclic or polycyclic group containing 3 to 20 carbon atoms. A 3-12 membered cycloalkyl is preferred. The term “monocyclic cycloalkyl” is preferably a 3-10 membered monocyclic cycloalkyl, more preferably a 3-8 membered monocyclic cycloalkyl, for example: cyclopropyl, cyclobutyl, cyclopentyl, cyclohexyl, cycloheptyl, cyclooctyl, cyclodecyl, cyclododecyl and cyclohexenyl. The term “polycyclic cycloalkyl” includes “bridged cycloalkyl”, “fused cycloalkyl” and “spirocycloalkyl”. Representative examples of “bridged cycloalkyl” include, but are not limited to: bornyl, bicyclo [2.2 .1]heptenyl, bicyclo [3.1.1]heptyl, bicyclo [2.2.1]heptyl, bicyclo [2.2.2]octyl, bicyclo [3.2.2] nonyl, bicyclo[3.3.1]nonyl, bicyclo[4.2.1]nonyl, adamantyl and the like. The term “fused cycloalkyl” includes cycloalkyl fused to phenyl, a cycloalkyl or a heteroaryl. The fused cycloalkyl includes, but is not limited to: benzocyclobutenyl, 2,3-dihydro-1-H-indenyl, 2,3-cyclopentenopyridinyl, 5,6-dihydro-4H-cyclopentyl[b]thiophenyl, decahydronaphthalenyl and the like. Representative examples of “spirocycloalkyl” include, but are not limited to: spiro[2,4]heptyl, spiro[4,5]decyl, and the like. The monocyclic cycloalkyl or polycyclic cycloalkyl can be linked to the parent molecule through any ring carbon atom.

The term “heterocycloalkyl” refers to a saturated or partially unsaturated (containing 1 or 2 double bonds) non-aromatic cyclic group consisting of carbon atoms and heteroatoms selected from nitrogen, oxygen, sulfur and the like. The cyclic group may be a monocyclic or polycyclic group. In the present invention, the number of heteroatoms in the heterocycloalkyl is preferably 1, 2, 3 or 4. The nitrogen, carbon or sulfur atoms in the heterocycloalkyl may optionally be oxidized. The nitrogen atom may optionally be further substituted with other groups to form tertiary amines or quaternary ammonium salts. More preferably, the heterocycloalkyl may be a 3-10 membered heterocycloalkyl. The “monocyclic heterocycloalkyl” is preferably a 3-10 membered monocyclic heterocycloalkyl, more preferably a 3-8 membered monocyclic heterocycloalkyl, for example, aziridinyl, tetrahydrofuran-2-yl, morpholin-4-yl, thiomorpholin-4-yl, thiomorpholin-S-oxide-4-yl, piperidin-1 -yl, N-alkylpiperidin-4-yl, pyrrolidin-l-yl, N-alkylpyrrolidin-2-yl, piperazin-l-yl, or 4-alkylpiperazin-1-yl. The term “polycyclic heterocycloalkyl” includes “fused heterocycloalkyl”, “spiroheterocycloalkyl”, and “bridged heterocycloalkyl”. “Fused heterocycloalkyl” includes a monocyclic heterocycloalkyl rings fused to phenyl, a cycloalkyl, a heterocycloalkyl or a heteroaryl, including but not limited to: 2,3-dihydrobenzofuranyl, 1,3 -dihydrois obenzofuranyl, indolinyl, 2,3 -dihydrobenzo [b]thienyl, dihydrobenzopyranyl, 1,2,3,4-tetrahydroquinolyl, and the like. The monocyclic heterocycloalkyl and polycyclic heterocycloalkyl can be linked to the parent molecule through any ring atom. Specifically, the above-mentioned ring atom refers to a carbon atom and/or a nitrogen atom constituting the ring backbone.

The term “cycloalkylalkyl” refers to a cycloalkyl linked to the parent structure through an alkyl . Thus, “cycloalkylalkyl” includes the definitions of alkyl and cycloalkyl above.

The term “heterocycloalkylalkyl” refers to a heterocycloalkyl linked to the parent structure through an alkyl . Thus, “heterocycloalkylalkyl” includes the definitions of alkyl and heterocycloalkyl above.

The term “aryl” refers to any stable 6-10 membered monocyclic or bicyclic aromatic group, for example: phenyl, naphthyl, tetrahydronaphthyl, 2,3-indanyl and biphenyl.

The term “heteroaryl” refers to an aromatic cyclic group formed by substituting at least 1 ring carbon atom with a heteroatom selected from nitrogen, oxygen and sulfur, which may be a 5-12 membered heteroaryl, preferably, may be a 5-7 membered monocyclic structure or a 7-12 membered bicyclic structure, preferably a 5-6 membered heteroaryl. In the present invention, the number of heteroatoms is preferably 1, 2 or 3, and the heteroaryl includes: pyridinyl, pyrimidinyl, pyridazin-3(2H)-onyl, furanyl, thienyl, thiazolyl, pyrrolyl, imidazolyl, pyrazolyl, oxazolyl, isoxazolyl, 1,2,5-oxadiazolyl, 1,2,4-oxadiazolyl, 1,2,4-triazolyl, 1,2,3-triazolyl, tetrazolyl, indazolyl, isoindazolyl, indolyl, isoindolyl, benzofuranyl, benzothienyl, benzo [d][1,3]dioxolanyl, benzothiazolyl, benzoxazolyl, quinolyl, isoquinolyl, quinazolinyl and the like.

The term “arylalkyl” refers to an aryl linked to the parent structure through an alkyl . Thus, “arylalkyl” includes the definitions of alkyl and aryl above.

The term “heteroarylalkyl” refers to a heteroaryl linked to the parent structure through an alkyl. Thus, “heteroarylalkyl” includes the definitions of alkyl and heteroaryl above.

The term “acyl” refers to a —C(O)—R₇ group, including alkylacyl, cycloalkylacyl and arylacyl, wherein R₇ is independently selected from alkyl, cycloalkyl and aryl unsubstituted or independently substituted at any position with 1 to 3 groups selected from one or more of C₁₋₄ alkyl, halogen, nitro, trihalomethyl and C₁₋₃ alkoxy. The acyl includes, but is not limited to: acetyl, benzoyl, trifluoroacetyl and the like.

The term “amino” refers to —NH₂, and the term “alkylamino” refers to an amino in which at least one hydrogen atom is substituted with an alkyl, including, but not limited to: —NHCH₂ and —NHCH₂CH₃. Thus, “alkylamino” includes the definitions of alkyl and amino above.

The term “inert gas” includes noble gases such as nitrogen, helium and argon.

As used herein, “room temperature” refers to 15-30° C.

The term “prodrug” refers to a compound that can be converted to an active compound through in vivo metabolism. Prodrug are generally substances that are inactive or less active than the active parent compound, but may provide convenient operation or administration, or improved metabolic performance.

The term “solvate” refers to a solvent addition form containing a stoichiometric or non-stoichiometric amount of solvent. Some compounds tend to capture a fixed molar proportion of solvent molecules in the crystalline solid state, thus forming solvates. If the solvent is water, the solvate formed is a “hydrate”. If the solvent is ethanol, the solvate formed is an ethanolate. A hydrate are a compound formed by combination of one or more water molecules with a substance, wherein the state of the water molecule is H₂O, and such combination can form a hydrate containing one or more water molecules.

The term “isomer” refers to that the compound of formula (I) of the present invention may have an asymmetric center and racemates, racemic mixtures and individual diastereomers, all of which, including stereoisomers and geometric isomers, are included in the present invention. Among these, the “isomer” of the present invention is preferably a “stereoisomer”. In the present invention, when the compound of formula (I) or a salt thereof is present in a stereoisomeric form (e.g., containing one or more asymmetric carbon atoms), individual stereoisomers (enantiomers and diastereomers) and mixtures thereof are included within the scope of the present invention. The present invention also includes individual isomers of the compound of formula (I) or a salt thereof, as well as mixtures with isomers in which one or more chiral centers are inverted. The scope of the present invention includes: mixtures of stereoisomers, and purified enantiomers or enantiomer/diastereomer-enriched mixtures. The present invention includes mixtures of stereoisomers in all possible combinations of any enantiomer and diastereomer. The present invention includes all combinations and subsets of stereoisomers of any specific groups defined above. The present invention also includes geometric isomers, including cis-trans isomers, of the compound of formula (I) or the salt thereof.

The above preferred conditions may be combined arbitrarily to obtain preferred embodiments of the present invention without departing from the general knowledge in the art. In the present invention, the compounds disclosed also include isotopically labeled compounds, which are identical to those of formula I, but have one or more atoms substituted by an atom having an atomic mass or mass number different from the atomic mass or mass number of those usually found in nature.

Examples of isotopes that can be incorporated into the compound of the present invention include isotopes of H, C, N, O, S, F and Cl, such as ²H, ³H, ¹³C, ¹¹C, ¹⁴C, ¹⁵N, ¹⁸O, ¹⁷O, ³²P, ³⁵S, ¹⁸F and ³⁶Cl.

The compounds of the present invention containing the aforementioned isotopes and/or other isotopes of other atoms, prodrugs thereof, or pharmaceutically acceptable salts of the compounds or the prodrugs are within the scope of the present invention. Certain isotopically labeled compounds of the present invention, for example those into which radioactive isotopes such as ³H and ¹⁴C are incorporated, are useful in tissue distribution assays of the drugs and/or substrates. Tritium (i.e., ³H) and carbon 14 (i.e., ¹⁴C) isotopes are particularly preferred due to ease of preparation and detectability. Furthermore, substitution with heavier isotopes such as deuterium (i.e., ²H) may afford certain therapeutic advantages (e.g., increased in vivo half-life or reduced dose) resulting from greater metabolic stability and hence may be preferred in some circumstances. The compounds of the present invention as claimed may be particularly limited to substitution by deuterium or tritium. Furthermore, the presence of hydrogen in a substituent where the terms deuterium or tritium are not separately stated does not suggest exclusion of deuterium or tritium, but may possibly include deuterium or tritium.

### DETAILED DESCRIPTION

The present invention is further illustrated by the following examples; however, these examples should not be construed as limiting the present invention. Experimental procedures without specified conditions in the following examples can be selected in accordance with conventional procedures and conditions. In the following examples, all the starting/auxiliary materials or reagents are commercially available unless otherwise specified.

The “nitrogen atmosphere” condition of the invention can be replaced by other inert gas atmosphere, such as “argon atmosphere”.

The structure of the compounds of the present invention can be identified by nuclear magnetic resonance (¹H NMR) and/or mass spectrometry (MS).

¹H NMR chemical shifts (6) were recorded in PPM (10⁻⁶). NMR was performed on a Bruker AVANCE-400 spectrometer. Suitable solvents are deuterated chloroform (CDCl₃), deuterated methanol (MeOD-d₄), deuterated dimethyl sulfoxide (DMSO-d₆) and tetramethylsilane (TMS) as internal standard. Liquid chromatography-mass spectrometry (LCMS) was conducted by an Agilent 1200HPLC/6120 mass spectrometer using XBridge C18, 4.6×50 mm, 3.5 μm. Gradient elution conditions one: 80-5% solvent A₁ and 20-95% solvent B₁ (1.8 minutes), followed by 95% solvent B₁ and 5% solvent A₁ (more than 3 minutes), the percentages being the volume percentage of a certain solvent in the total solvent volume. Solvent A₁: 0.01% trifluoroacetic acid (TFA) in water; solvent B₁: 0.01% trifluoroacetic acid in acetonitrile; the percentages being the volume percent of solute in solution. Gradient elution conditions two: 80-5% solvent A₂ and 20-95% solvent B₂ (1.5 minutes), followed by 95% solvent B₂ and 5% solvent A₂ (more than 2 minutes), the percentages being the volume percentage of a certain solvent in the total solvent volume. Solvent A₂: 10 mM aqueous ammonium bicarbonate; solvent B₂: acetonitrile.

The compounds of the present invention can be separated and purified by using conventional column chromatography, flash separator or high-performance liquid chromatography, and the elution system can be an ethyl acetate/petroleum ether system or a dichloromethane/methanol system.

The fast separator (flash column chromatography; flash system/Cheetah™) was equipped with Agela Technologies MP200, and the flash chromatographic column was Flash column Silica-CS (80 g; Cat No. CS140080-0).

The preparative high-performance liquid chromatograph (prep-HPLC) was equipped with Shimadzu LC-20, and the column was Waters Xbridge Pre C18, 10 μm, 19 mm×250 mm. Mobile phase A: 0.05% trifluoroacetic acid in water (percentage being a volume percent), mobile phase B: acetonitrile; detection wavelength: 214 nm & 254 nm; flow rate: 15.0 mL/min.

The column chromatography generally used 200-mesh and 300-mesh silica gel (Huanghai, Yantai) as the resin. The thin layer chromatograph (TLC) was equipped with HSGF254 (Huanghai, Yantai) or GF254 (Qingdao) silica gel plate.

### EXAMPLES 1-3

### Preparation of compound of formula 1-2

### EXAMPLE 1

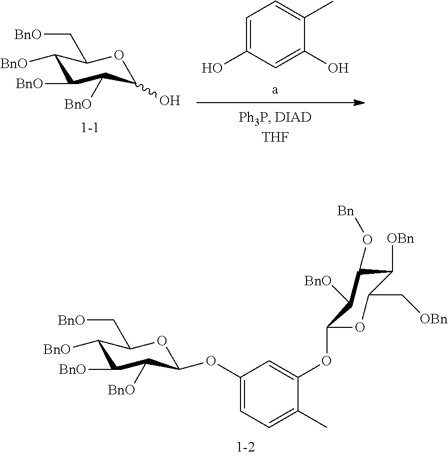

A solution of triphenylphosphine (52.82 g), compound a (10 g, 1 eq.) and a compound of formula 1-1 (91.46 g) in anhydrous tetrahydrofuran (TRF; 1000 mL) was cooled to −15° C. in an ice bath. DIAD (diisopropylazodicarboxylate; 40.72 g, 2.5 eq.) was slowly and dropwise added to the solution, and the solution gradually turned yellow. After 30 minutes of stirring at 0° C., the ice bath was removed. The reaction system was warmed to room temperature (25° C.) and continuously stirred until the starting material disappeared (TLC monitoring, about 6 hours).

After the reaction was completed, the solution was concentrated in vacuum. The product was separated and purified by silica gel column chromatography to give a white foamy solid (53% yield). Rf 0.37 (EtOAc/hexane =1/5); 1H NMR (600 MHz, CDCl₃,) δ 7.25-7.05 (m, 40H), 6.98-6.95 (m, 1H) 6.78-6.74 (m, 1H) 6.65-6.55 (m, 1H), 5.05-4.89 (m, 4H), 4.85-4.62 (m, 8H), 4.55-4.40 (m, 6H), 4.71-4.45 (m, 12H) 2.16 (s, 3H).

### EXAMPLE 2

A solution of tributylphosphine (40.75 g), compound a (10 g, 1 eq.) and a compound of formula 1-1 (91.46 g) in dichloromethane (800 mL) was cooled to −10° C. in an ice bath. Dimethyl azodicarboxylate (DMAD, 61.79 g, 2.5 eq.) was slowly and dropwise added to the solution, and the solution gradually turned yellow. After 40 minutes of stirring at 0° C., the ice bath was removed. The reaction system was warmed to 50° C. and continuously stirred until the starting material disappeared (TLC monitoring, about 5 hours).

After the reaction was completed, the solution was concentrated in vacuum. The product was separated and purified by silica gel column chromatography to give a white foamy solid (56% yield). Rf 0.37 (EtOAc/hexane=1/5); 1H NMR (600 MHz, CDCl₃,) δ7.25-7.05 (m, 40H), 6.98-6.95 (m, 1H) 6.78-6.74 (m, 1H) 6.65-6.55 (m, 1H), 5.05-4.89 (m, 4H), 4.85-4.62(m, 8H), 4.55-4.40 (m, 6H), 4.71-4.45 (m, 12H) 2.16 (s, 3H).

### EXAMPLE 3

A solution of trimethylphosphine (15.32 g), compound a (10 g, 1 eq.) and a compound of formula 1-1 (91.46 g) in toluene (800 mL) was cooled to 0° C. in an ice bath. Diethyl azodicarboxylate (DEAD, 73.65g, 2.5 eq.) was slowly & dropwise added to the solution, and the solution gradually turned yellow. After 20 minutes of stirring at 0° C., the ice bath was removed. The reaction system was warmed to 20° C. and continuously stirred until the starting material disappeared (TLC monitoring, about 6 hours). After the reaction was completed, the solution was concentrated in vacuum. The product was separated and purified by silica gel column chromatography to give a white foamy solid (50% yield).

Rf 0.37 (EtOAc/hexane=1/5); 1H NMR (600 MHz, CDCl₃,) δ7.25-7.05 (m, 40H), 6.98-6.95 (m, 1H) 6.78-6.74 (m, 1H) 6.65-6.55 (m, 1H), 5.05-4.89 (m, 4H), 4.85-4.62 (m, 8H), 4.55-4.40 (m, 6H), 4.71-4.45 (m, 12H) 2.16 (s, 3H).

### EXAMPLES 4-6

### Preparation of compound of formula 1

### EXAMPLE 4

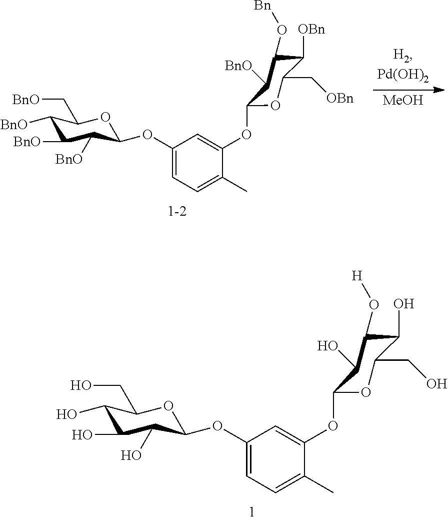

The compound of formula 1-2 (47 g) was dissolved in methanol (1000 mL). A palladium hydroxide on activated carbon catalyst (22.58 g, containing 20 wt % of palladium hydroxide) was added in nitrogen atmosphere and the reaction system was stirred at 45° C. in hydrogen atmosphere at one standard atmosphere pressure until complete conversion of the starting materials (TLC monitoring, about 12 hours). The reaction solution was filtered under reduced pressure. The filter cake was washed with methanol (3×200 mL). The filtrates were combined and the solvent was removed under reduced pressure to give a product in the form of a white powder (16 g, 88% yield).

Rf 0.37 (MeOH/CH₂Cl₂=1/5);¹H NMR (600 MHz, CDCl₃,) δ7.14-7.13 (d, 1H), 6.80 (s, 1H) 6.73-6.71 (d, 1H) 5.00-4.98 (m, 2H),3.88-3.85 (m, 2H), 3.68-3.60 (m, 2H), 3.60-3.40 (m, 8H), 2.13 (s, 3H); ¹³C NMR (600 MHz, CDCl₃,) δ155.5, 155.1, 131.4, 122.3, 110.6, 103.8, 100.2, 76.2, 75.5, 72.8, 69.5, 60.7, 48.8, 14.6;IR (thin film,cm-1): 3257, 2928, 1612, 1592, 1503, 1395, 1264, 1169, 1058, 922, 896;HRMS (ESI-TOF) m/z Calcd.for C₁₉H₂₈O₁₂: [M+Na]⁺471.1478, found 471.1484;[α]²³_{D}=−74.0° (c 1, H₂O).

### EXAMPLE 5

The compound of formula 1-2 (47 g) was dissolved in ethanol (1000 mL). A palladium on carbon catalyst (5.0 g, containing 10 wt % of Pd) was added in nitrogen atmosphere and the reaction system was stirred at 20° C. in hydrogen atmosphere at one standard atmosphere pressure until complete conversion of the starting materials (TLC monitoring, about 13 hours). The reaction solution was filtered under reduced pressure. The filter cake was washed with methanol (3×200 mL). The filtrates were combined and the solvent was removed under reduced pressure to give a product in the form of a white powder (15.5 g, 85.2% yield).

Rf 0.37 (MeOH/CH₂Cl₂=1/5);¹H NMR (600 MHz, CDCl₃,) δ7.14-7.13 (d, 1H), 6.80 (s, 1H) 6.73-6.71 (d, 1H) 5.00-4.98 (m, 2H),3.88-3.85 (m, 2H), 3.68-3.60 (m, 2H), 3.60-3.40 (m, 8H), 2.13 (s, 3H);¹³C NMR (600 MHz, CDCl₃,) δ155.5, 155.1, 131.4, 122.3, 110.6, 103.8, 100.2, 76.2, 75.5, 72.8, 69.5, 60.7, 48.8, 14.6;IR (thin film,cm-1): 3257, 2928, 1612, 1592, 1503, 1395, 1264, 1169, 1058, 922, 896;HRMS (ESI-TOF) m/z Calcd. for C₁₉H₂₈O₁₂: [M+Na]⁺471.1478, found 471.1484;[α]²³_{D}=−74.0° (c 1, H₂O).

### EXAMPLE 6

The compound of formula 1-2 (47g) was dissolved in isobutanol (1000 mL). A palladium hydroxide on activated carbon catalyst (22.58 g, containing 20 wt % of palladium hydroxide) was added in nitrogen atmosphere and the reaction system was stirred at 50° C. in hydrogen atmosphere at one standard atmosphere pressure until complete conversion of the starting materials (TLC monitoring, about 12 hours). The reaction solution was filtered under reduced pressure. The filter cake was washed with methanol (3×200 mL). The filtrates were combined and the solvent was removed under reduced pressure to give a product in the form of a white powder (90% yield).

Rf 0.37 (MeOH/CH₂Cl₂=1/5);¹H NMR (600 MHz, CDCl₃,) δ7.14-7.13 (d, 1H), 6.80 (s, 1H) 6.73-6.71 (d, 1H) 5.00-4.98 (m, 2H),3.88-3.85 (m, 2H), 3.68-3.60 (m, 2H), 3.60-3.40 (m, 8H), 2.13 (s, 3H);¹³C NMR (600 MHz, CDCl₃,) δ155.5, 155.1, 131.4, 122.3, 110.6, 103.8, 100.2, 76.2, 75.5, 72.8, 69.5, 60.7, 48.8, 14.6;IR (thin film,cm-1): 3257, 2928, 1612, 1592, 1503, 1395, 1264, 1169, 1058, 922, 896;HRMS (ESI-TOF) m/z Calcd. for C₁₉H₂₈O₁₂: [M+Na]⁺471.1478, found 471.1484;[α]²³_{D}=−74.0° (c 1, H₂O).

### EXAMPLE 7

### Efficacy of Oral Administration for Compound of Formula 1 in Treating Depression

(1) Experimental Animals

The experimental animals were male SPF grade KM mice purchased from Kunming Medical University, weighed 21-24 g, certificate no. SCXK(Dian)K2015-0002. The experimental animals were bred in individually ventilated cages (IVCs) in animal room in Dianqing Biotechnology, Ltd., Yunnan (facility no. 13-11-078 and 13-11-079; manufacture date: Nov. 24, 2013). The room temperature was controlled at 22-24° C. with humidity at 40-70% and 12-hour light/dark cycle (7:00 am/19:00 pm). The cages and padding were changed twice a week. The raising method was group raising and there were 10 mice accommodated in each cage. The feed was sterilized feed from Jiangsu Xietong Medical Bioengineering, Ltd., certificate no.: (2014)01008. Feed was supplied once daily with free access . Tap water was supplied in boxes with free access.

(2) Test Compound

The test compound was the compound of formula 1 (product in Example 4), with a molecular weight of 448.16, which is easily soluble in water and was sealed at 4° C.

(3) Experimental Procedures

Grouping:

A. Behavioral testing was performed in different treatment groups (1.0 mg/kg, 5.0 mg/kg, 10.0 mg/kg, 15.0 mg/kg, 30.0 mg/kg) 1 h after intragastric (i.g.) administration of the compound of formula 1;

B. In the negative control group receiving normal saline (NS), the behavioral testing was conducted at the same time;

C. In the positive control group receiving imipramine (IMI; 15 mg/kg), the behavioral testing was conducted at the same time;

Route of administration: The test compound of formula 1, normal saline or imipramine was administered by intragatric administration according to the experimental design.

Time of administration: The animals were accommodated to the experimental environment for 1 hour, administrated by intragatric administration, and subjected to tail suspension test 1 hour after the administration.

Test criterion: The mice were subjected to a 6-minute tail suspension test, and the cumulative time of immobility in the first 2 minutes and the last 4 minutes was recorded. The criterion for immobility is that the mouse stops struggling and remains steady.

(4) Procedures

A depression model of behavioral despair, ie. tail suspension model, was used. Animals were accommodated to the company's breeding environment for 1 day. During the adaptation, animals with non-smooth and unclean hair, high alertness or aggressivity were excluded.

Animals were accommodated to the experimental environment for 1 hour, weighed, and randomized according to the body weight into a normal saline control group, an imipramine control group and treatment groups with different doses.

In the tail suspension test, animals in all groups were administered with corresponding drugs by a single intragastric administration. 1 h after the administration, tails of mice were fixed with medical tape at about 1-2 cm from the end, such that the mice were hanged in the tail suspension box with the heads about 10 cm above the bottom of the box. The observation started immediately after hanging. In the 6-minute observation, the cumulative time of immobility in the first 2 minutes and the last 4 minutes was recorded. A video was recorded with obviously contrasted background with the hair color of the mice, for example, black background was used for white mice.

(5) Statistics

The cumulative time of immobility within the last 4 minutes was compared between the test compound groups and the normal saline group using SPSS 11.0 software. One-way analysis of variance (ANOVA) was used for comparison among multiple groups, and independent sample T test was performed for comparisons in pairs. P <0.05 indicates a statistically significant difference. All statistical diagrams were plotted in mean ±SEM using Origin 8.0 software.

(6) Results

Mice were administered with different doses of the compound of formula 1 by intragastric administration, and subjected to a 6-minute tail suspension test 1 hours after the administration. The results show that: the time of immobility in each treatment group (1.0 mg/kg, 5.0 mg/kg, 10.0 mg/kg, 15.0 mg/kg, 30.0 mg/kg) of the compound of formula 1 was lower than that of the control group (100%). Among these, the time of immobility in 5.0 mg/kg, 10.0 mg/kg, 15.0 mg/kg treatment groups was very significantly different as compared to the normal saline group (**P <0.01); the time of immobility in the 30.0 mg/kg group was significantly different as compared to the normal saline group (*P <0.05). The compound of formula 1 is capable of significantly reducing the time of immobility in mice and has significant dose-response relationship. The detailed results are shown in Table 1.

TABLE 1
Efficacy of oral administration for compound of
formula 1 in treating depression

| Grouping | Dose (mg/kg, i.g.) | N | Time of immobility (% of NS) |
|---|---|---|---|
| NS | — | 69 | 100.00 ± 5.08 |
| IMI | 15 | 71 | 58.64 ± 5.42*** |
| Compounds | 1 | 30 | 87.14 ± 11.27 |
| of formula 1 | 5 | 29 | 72.67 ± 7.89** |
| | 10 | 25 | 66.86 ± 8.80** |
| | 15 | 31 | 68.33 ± 7.17** |
| | 30 | 29 | 77.38 ± 6.00* |

*P < 0.05; **P < 0.01; ***P < 0.001 (compared to NS), least significant difference test after one-way ANOVA.

Conclusion: the compound of formula 1 can significantly reduce the time of immobility in mice through oral administration, suggesting that the compound has anti-depression efficacy and significant dose-response relationship.

### EXAMPLE 8

### Efficacy of Intraperitoneal Injection for Compound of Formula 1 in Treating Depression

(1) Experimental Animals

The experimental animals were male SPF grade KM mice purchased from Kunming Medical University, weighed 21-24 g, certificate no. SCXK(Dian)K2015-0002. The experimental animals were bred in individually ventilated cages (IVCs) in animal room in Dianqing Biotechnology, Ltd., Yunnan (facility no. 13-11-078 and 13-11-079; manufacture date: Nov. 24, 2013). The room temperature was controlled at 22-24° C. with humidity at 40-70% and 12-hour light/dark cycle (7:00 am/19:00 pm). The cages and padding were changed twice a week. The raising method was group raising and there were 10 mice accommodated in each cage. The feed was sterilized feed from Jiangsu Xietong Medical Bioengineering, Ltd., certificate no.: (2014)01008. Feed was supplied once daily with free access. Tap water was supplied in boxes with free access.

(2) Test Compound

The test compound was the compound of formula 1 (product in Example 4), with a molecular weight of 448.16, which is easily soluble in water and was sealed at 4° C.

(3) Experimental Procedures

Grouping:

A. Behavioral testing was performed in different treatment groups (1.0 mg/kg, 5.0 mg/kg, 10.0 mg/kg, 20.0 mg/kg) 0.5 h after intraperitoneal (i.p.) injection of the compound of formula 1;

B. In the control group receiving normal saline (NS), the behavioral testing was conducted at the same time;

C. In the control group receiving imipramine (IMI; 15 mg/kg), the behavioral testing was conducted at the same time;

Route of administration: The test compound of formula 1, normal saline or imipramine was administered by intraperitoneal injection according to the experimental design.

Time of administration: The animals were accommodated to the experimental environment for 1 hour, administrated by intraperitoneal injection and subjected to tail suspension test 0.5 hour after the administration.

Test criterion: The mice were subjected to a 6-minute tail suspension test, and the cumulative time of immobility in the first 2 minutes and the last 4 minutes was recorded. The criterion for immobility was that the mouse stops struggling and remains steady.

(4) Procedures

A depression model of behavioral despair, ie. tail suspension model, was used. Animals were accommodated to the company's breeding environment for 1 day. During the adaptation, animals with non-smooth and unclean hair, high alertness or aggressivity were excluded.

Animals were accommodated to the experimental environment for 1 hour, weighed, and randomized according to the body weight into a normal saline control group, an imipramine control group and treatment groups with different doses.

In the tail suspension test, animals in all groups for tail suspension test were administered with corresponding drugs by a single intraperitoneal injection. 0.5 h after the administration, tails of mice were fixed with medical tape at about 1-2 cm from the end, such that the mice were hanged in the tail suspension box with the heads about 10 cm above the bottom of the box. The observation started immediately after hanging. In the 6-minute observation, the cumulative time of immobility in the first 2 minutes and the last 4 minutes was recorded. A video was recorded with obviously contrasted background with the hair color of the mice, for example, black background was used for white mice.

(5) Statistics

The cumulative time of immobility within the last 4 minutes was compared between the test compound groups and the normal saline group using SPSS 11.0 software. One-way analysis of variance (ANOVA) was used for comparison among multiple groups, and independent sample T test was performed for comparisons in pairs. P <0.05 indicates a statistically significant difference. All statistical diagrams were plotted in mean ±SEM using Origin 8.0 software.

(6) Results

Mice were administered with different doses of the compound of formula 1 by intraperitoneal injection, and subjected to a 6-minute tail suspension test 30 minutes after the administration. The results show that: the time of immobility in 5.0 mg/kg and 10.0 mg/kg treatment groups (1.0 mg/kg, 5.0 mg/kg, 10.0 mg/kg, 20.0 mg/kg) of the compound of formula 1 was significantly lower than that of the control group. Among these, the time of immobility in 5.0 mg/kg treatment group was significantly different as compared to the normal saline group (**P <0.05); the time of immobility in the 10.0 mg/kg group was very significantly different as compared to the normal saline group (*P <0.01). The intraperitoneal injection of the compound of formula 1 is capable of significantly reducing the time of immobility in mice and has significant dose-response relationship. The detailed results are shown in Table 2.

TABLE 2
Efficacy of intraperitoneal injection for compound of
formula 1 in treating depression

| Grouping | Dose (mg/kg, i.p.) | N | Time of immobility (% of NS) |
|---|---|---|---|
| NS | — | 28 | 100.00 ± 5.95 |
| IMI | 15 | 31 | 58.12 ± 5.66*** |
| Compounds | 1 | 14 | 104.02 ± 16.80 |
| of formula 1 | 5 | 24 | 72.12 ± 8.57* |
| | 10 | 24 | 60.44 ± 7.53** |
| | 20 | 14 | 104.66 ± 14.75 |

*P < 0.05; **P < 0.01; ***P < 0.001 (compared to NS), least significant difference test after one-way ANOVA.

Conclusion: the compound of formula 1 can significantly reduce the time of immobility in mice through intraperitoneal injection, suggesting that the compound has significant dose-response relationship.

### EXAMPLE 9

### Positive Regulation of NMDA Receptor Current by Compound of Formula 1

(1) Experimental Animals

Male C57BL/6 mice, aged 3-9 weeks, were purchased from Beijing Vital River Laboratory Animal Technology Co., Ltd. (Beijing, China). The animals were bred at 23 ±1° C. The room temperature was controlled at 22-24° C. with humidity at 40-70% and 12-hour light/dark cycle (7:00 am/19:00 pm).

The cages and padding were changed twice a week. The raising method was group raising and there were 3-5 mice accommodated in each cage. Feed was supplied once daily with free access. Tap water was supplied in boxes with free access.

(2) Test Compound

The test compound was the compound of formula 1 (product in Example 4), with a molecular weight of 448.16, which is easily soluble in water and was sealed at 4° C.

(3) Experimental Procedures

Grouping:

A. The baseline whole-cell NMDAR current was recorded in 10 minutes before the compound of formula 1 (0.76, 3.8 mM/L) was dissolved in artificial cerebrospinal fluid (vehicle) and added with the brain slice circulation fluid.

B. An identical volume of artificial cerebrospinal fluid (vehicle) was added with the brain slice circulation fluid.

Treatment: The brain slice circulation fluid was directly added.

Time of treatment: 10 minutes after baseline recording.

Test item: depolarization voltage-induced whole-cell NMDAR receptor currents.

Hippocampal brain slices: The animals were anesthetized with isoflurane and the brains were collected quickly. Brain slices with a thickness of 350 micron were prepared with a vibrating blade microtome (Leica VT1000S, Leica Microsystems, Germany) and incubated in artificial cerebrospinal fluid with saturated oxygen (95% 02/5% CO₂) on ice: 206 mM sucrose, 2.5 mM KCl, 1.25 mM NaH2PO₄, 26 mM NaHCO₃, 10 mM D-glucose, 2 mM MgSO₄.7H₂O and 2 mM CaCl₂.H₂O (pH 7.2-7.4, 290-300 mOsm). Subsequently, the brain slices were incubated at 32° C. for another 45 minutes in artificial cerebrospinal fluid with saturated oxygen (95% 02/5% CO₂). Finally, the brain slices were transferred to a recording tank containing continuously circulating artificial cerebrospinal fluid with saturated oxygen.

Whole-cell NMDAR current recording: NMDAR whole cell EPSC recording was performed in hippocampal CA1 pyramidal cells. Recording electrodes were made with a micropipette puller (P-1000, Sutter, USA) with an input resistance of about 5-7 MQ. The filling electrode solution: 130 mM Cs-methanesulfonate, 0.15 mM CaCl₂.2H₂O, 2.0 mM MgC12, 2.0 mM EGTA, 10 mM HEPES, 2 mM Mg-ATP, 0.3 mM Na-GTP, and 10 mM QX-314 with osmolarity adjusted to 285-290 mOsm/kg and pH adjusted to 7.2 with CsOH. Hippocampal pyramidal cells were clearly visible under a 40-fold water microscope and near infrared visual system (Olympus, BX51WI, Japan). The electrical signal in whole cells was recorded using a Clampfit 10.3 software (Axon Instruments) equipped with an Axopatch-700B amplifier (Axon Instruments, Foster City, Calif.) and a Digiclata 1440A digital-to-analog converter, with filtering set at 2.8 kHz and sampling at 10 kHz. Hippocampal CA1 pyramidal cells were clamped at a membrane potential of +40 mV. Schaffer collaterals were stimulated using a white iraurita electrode, triggering glutamate release resulting in whole cell NMDAR-mediated EPSC. The NIVIDAR-EPSC were validated with antagonist AP-5 of NMDARs. After 10 minutes of NMDAR-EPSC recording (once every 20 seconds), vehicle or the compound of formula 1 was added to the circulating artificial cerebrospinal fluid for another 20 minutes, and the NMDAR-EPSC current intensity (pA) was measured for the amplitude of the last 10 minutes of recorded EPSC and the amplitude of baseline.

(4) Statistics

The data were compared between the test compound groups and the vehicle group using SPSS 11.0 software. One-way analysis of variance (ANOVA) was used for comparison among multiple groups, and independent sample T test was performed for comparisons in pairs. P <0.05 indicates a statistically significant difference. All statistical diagrams were plotted in mean ±SEM using Origin 8.0 software.

(5) Results

Addition of vehicle to oxygenated circulating cerebrospinal fluid had no effect on NMDAR-EPSC, giving a result of 98.31 ±3.28% compared to the baseline. However, the addition of 0.76 mM/L of the compound of formula 1 slightly increased NMDAR-EPSC to 108.80 ±5.86% compared to baseline, which, however, is statistically insignificant. However, the addition of 3.8 mM/L of the compound of formula 1 significantly increased NMDAR-EPSC to 155.53 ±20.85% compared to baseline, which is statistically significant (**P <0.01). The detailed results are shown in Table 3.

TABLE 3
Positive regulation of NMDA receptor current
by compound of formula 1

| | | | NMDAR current |
|---|---|---|---|
| Grouping | Dose (mM/L) | N | (% of Baseline) |
| Vehicle | — | 5 | 98.31 ± 3.28 |
| Compounds of | 0.76 | 6 | 108.80 ± 5.86 |
| formula 1 | 3.8 | 4 | 155.53 ± 20.85** |

**P < 0.01 (compared to Vehicle), least significant difference test after one-way ANOVA.

Conclusion: The compound of formula 1 can directly up-regulate NMDA receptor function and has a dose-response relationship.

1. An aromatic ring compound of formula I, an isomer, a prodrug, a solvate, a pharmaceutically acceptable salt or an isotopically labeled compound thereof, 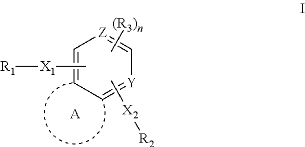
wherein,
R₁ and R₂ each independently represent H or a saccharide unit, and at least one of R₁ and R₂ is a saccharide unit; the saccharide unit may be selected from C₄₋₆ monosaccharides such as glucose, mannose, allose, galactose, arabinose and xylose, or may be selected from disaccharides and higher-order oligosaccharides such as sucrose, lactose, cellobiose and maltose, wherein carbon and oxygen atoms on the saccharide unit may be optionally substituted with sulfur, nitrogen or carbon;
when R₁ and R₂ each independently represent H, the —X₁— and —X₂— to which they are connected respectively represent —O—, —S— or a bond;
when R₁ and R₂ each independently represent a saccharide unit, the —X₁— and —X₂— to which they are connected respectively represent glycosidic bond formed by the saccharide unit and a non-saccharide unit (aromatic aglycon) and each independently represent —O—, —O—, —N— or a bond (i.e., O-glycosidic bond, S-glycosidic bond, N-glycosidic bond, or C-glycosidic bond is formed); or —X₁— and —X₂— are —CH₂—;
Y and Z each independently represent C, O, N, S, P or Si;
R₃ represents hydrogen, hydroxyl, or a substituted or unsubstituted C₁-C₂₀ aliphatic hydrocarbyl;
n is selected from 1, 2, 3, 4 and 5; the aromatic ring may be 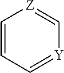
(with absence of ring A) or 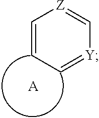
ring A may be a C₆₋₁₀ aryl, a C₃₋₈ cycloalkyl, a 3-10 membered heterocycloalkyl, or a 5-12 membered heteroaryl.

2. The aromatic ring compound, the isomer, the prodrug, the solvate, the pharmaceutically acceptable salt or the isotopically labeled compound thereof according to claim 1, wherein ring A may be phenyl ring, a 5-6 membered heteroaryl, a C₅₋₆ cycloalkyl or a 5-6 membered heterocycloalkyl; in ring A, a heteroatom, if present, may be O, S or N; ring A may be, for example, phenyl ring, cyclopentane, cyclohexane, or a nitrogen- or oxygen-containing 5-6-membered heterocyclic ring;
and/or, the C₁-C₂₀ aliphatic hydrocarbyl may be a saturated hydrocarbyl or an unsaturated hydrocarbyl, for example, selected from a C₁-C₂₀ alkyl, a C₂-C₂₀ alkenyl and a C₂-C₂₀ alkynyl, and specifically, selected from a (C₁-C₆) alkyl, a (C₂-C₆) alkenyl and a (C₂-C₆) alkynyl;
and/or, the substituted C₁-C₂₀ aliphatic hydrocarbyl may be a C₁-C₂₀ aliphatic hydrocarbyl containing one, two or more halogen and/or oxygen, sulfur, nitrogen, phosphorus atoms; for example, a halogenated (C₁-C₆) alkyl, a halogenated (C₁-C₆) alkoxy, or a (C₁-C₆) alkoxy, and specifically, CF₃, CHF₂, and OCH₃; for example, a C₁-C₂₀ aliphatic hydrocarbyl substituted with hydroxyl, amino, carboxyl, fluorine, trifluoromethyl, difluoromethyl, formyl, or phosphate, sulfate, phosphate or sulfonate group; the halogen is selected from F, Cl, Br and I;
and/or, the saccharide unit is preferably glucose, mannose, allose, galactose, arabinose or xylose;
and/or, the saccharide unit may be in the D configuration or L configuration;
and/or the configurations of the glycosidic bonds formed by the saccharide unit and the aromatic aglycon are independently selected from an a configuration and a β configuration, preferably a β configuration;
and/or, the glycosidic bond may be formed by connecting the aglycon to the C1 position of the ring moiety of the saccharide unit; and/or, the aromatic ring may be phenyl ring, 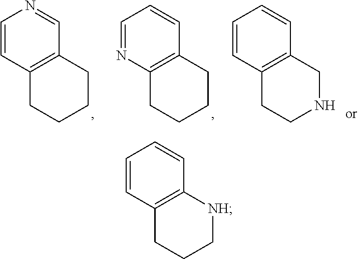
and/or, in the isotopically labeled compound, the isotopically labeled atoms include, but are not limited to, hydrogen, carbon, nitrogen, oxygen and phosphorus, as they can be substituted by isotopically labeled atoms ²H, ³H, ¹¹C, ¹³C, ¹⁴C, ¹⁵N, ³¹P, ³²P and ³⁵S.

3. The aromatic ring compound, the isomer, the prodrug, the solvate, the pharmaceutically acceptable salt or the isotopically labeled compound thereof according to claim 1, wherein when R₃ is a C₁-C₂₀ aliphatic hydrocarbyl containing amino functional group or the aromatic ring is a nitrogen-containing heterocyclic ring, the compound can form the pharmaceutically acceptable salt with an acid; preferably, the acid is selected from sulfuric acid, phosphoric acid, methanesulfonic acid, p-toluenesulfonic acid, benzenesulfonic acid, citric acid, oxalic acid, lactic acid, acetic acid, succinic acid, any one of the 20 natural L-amino acids and corresponding D-amino acids thereof, and an oxygen-free acid; the oxygen-free acid may be HCl, HBr, HI or HF.

4. The aromatic ring compound, or the isomer, the prodrug, the solvate, the pharmaceutically acceptable salt or the isotopically labeled compound thereof according to claim 1, wherein the aromatic ring compound is selected from:

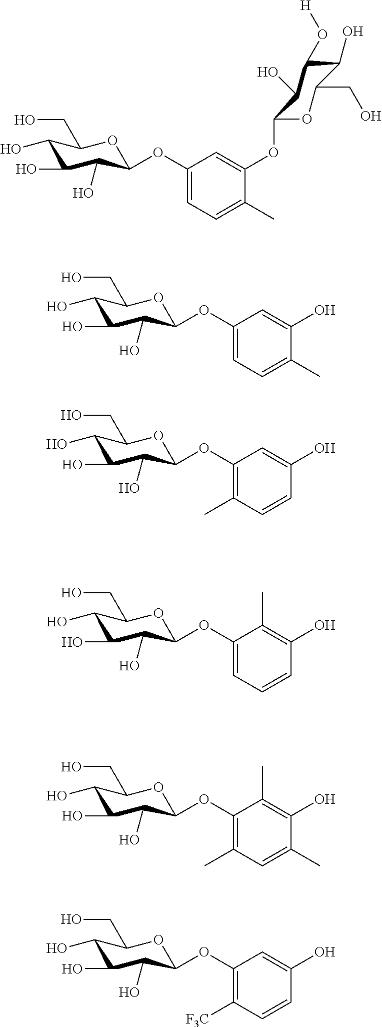

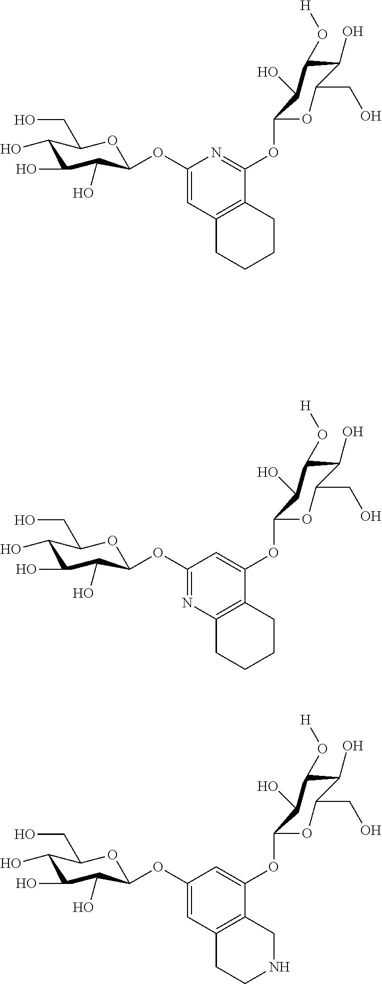

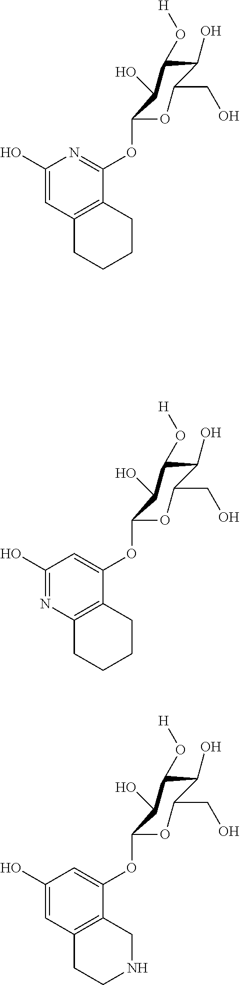

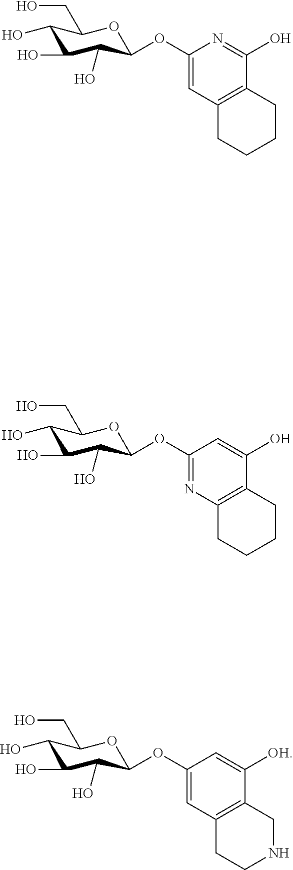

5. A pharmaceutical composition comprising the aromatic ring compound, the isomer, the prodrug, the solvate, the pharmaceutically acceptable salt or the isotopically labeled compound thereof according to claim 1, and a pharmaceutically acceptable carrier.

6. Use of the aromatic ring compound, the isomer, the prodrug, the solvate, the pharmaceutically acceptable salt or the isotopically labeled compound thereof according to claim 1, in preparing a medicament for treating depressive disorders.

7. The use according to claim 6, wherein the aromatic ring compound, the isomer, the prodrug, the solvate, the pharmaceutically acceptable salt or the isotopically labeled compound thereof is used alone or in combination with other therapeutic agents for treating nerve damage and depressive disorders.

8. A pharmaceutical formulation comprising the aromatic ring compound, the isomer, the prodrug, the solvate, the pharmaceutically acceptable salt or the isotopically labeled compound thereof according to claim 1; preferably, the formulation is selected from an injection, an oral capsule and tablet, and other conventional dosage forms.

9. A compound of the following formula:

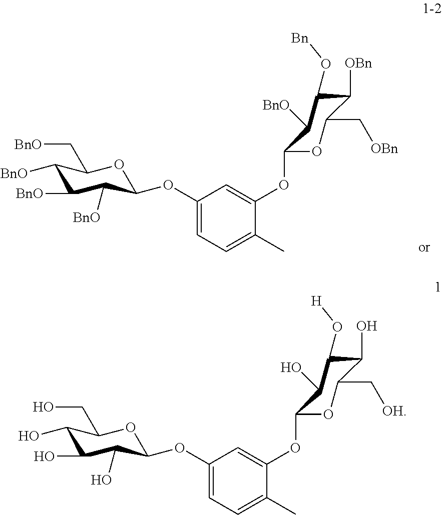

10. A method for preparing the aromatic ring compound, or the isomer, the prodrug, the solvate, the pharmaceutically acceptable salt or the isotopically labeled compound thereof according to claim 1, comprising: condensing a hydroxyl-protected saccharide starting material with an aglycon, followed by deprotecting to give a product; wherein, the method further may comprise a post-treatment procedure; preferably, the reaction scheme is as follows: 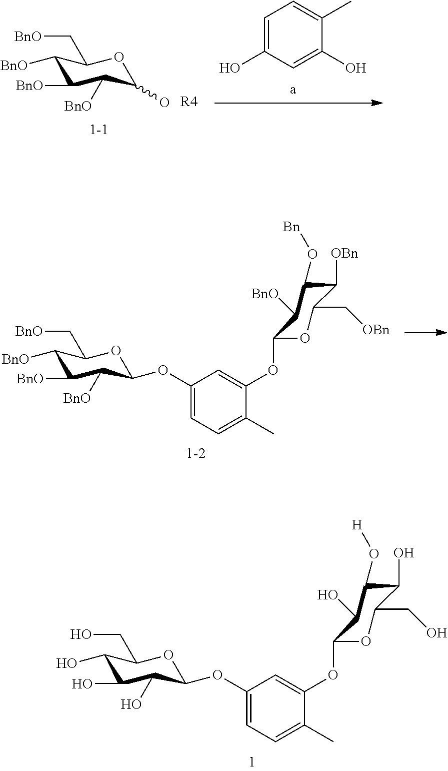
wherein R₄ is selected from hydrogen and an isotopically-labeled atom thereof;
the preparation method comprises the following procedures:
1) subjecting a compound of formula 1-1 and compound a to Mitsunobu reaction to give a compound of formula 1-2; and
2) subjecting the compound of formula 1-2 to catalytic hydrogenolysis reaction to give a compound of formula 1.

11. A method for treatment of depressive disorders, comprising administering a therapeutically effective amount of the aromatic ring compound, the isomer, the prodrug, the solvate, the pharmaceutically acceptable salt or the isotopically labeled compound thereof according to claim 1.

12. The method of claim 11, wherein the aromatic ring compound, the isomer, the prodrug, the solvate, the pharmaceutically acceptable salt or the isotopically labeled compound thereof according claim 1 is administered alone or in combination with other therapeutic agents.
